(19) United States
(12) Patent Application Publication
(10) Pub. No.: US 2023/0354897 A1
(43) Pub. Date: Nov. 9, 2023

(54) CERAMIC SUBSTRATE, CERAMIC HEATING BODY, AND ELECTRONIC VAPORIZATION DEVICE

(71) Applicant: SHENZHEN SMOORE TECHNOLOGY LIMITED, Shenzhen (CN)

(72) Inventors: Zhichao CHEN, Shenzhen (CN); Xianjun FU, Shenzhen (CN); Yue JIANG, Shenzhen (CN)

(21) Appl. No.: 18/357,072

(22) Filed: Jul. 21, 2023

Related U.S. Application Data
(63) Continuation of application No. PCT/CN2021/142003, filed on Dec. 28, 2021.

(30) Foreign Application Priority Data
Jan. 27, 2021 (WO) PCT/CN2021/073998

(51) Int. Cl. A24F 40/46; A24F 40/485; A24F 40/10

(52) U.S. Cl. CPC A24F 40/46; A24F 40/485; A24F 40/10

(57) ABSTRACT

A ceramic substrate is provided. A thickness of the ceramic substrate ranges from 1 to 4 mm, and a thermal conductivity of the ceramic substrate ranges from 0.8 to 2.5 W/m·k. In an embodiment, the thickness of the ceramic substrate ranges from 1.5 to 3 mm. In an embodiment, the thermal conductivity of the ceramic substrate ranges from 1.0 to 2.0 W/m·k.

### CROSS-REFERENCE TO PRIOR APPLICATION

This application is a continuation of International Patent Application No. PCT/CN2021/142003, filed on Dec. 28, 2021, which claims priority to International Patent Application No. PCT/CN2021/073998, filed on Jan. 27, 2021. The entire disclosure of both applications is hereby incorporated by reference herein.

### FIELD

This application relates to the technical field of electronic cigarettes, and specifically, to a ceramic substrate, a ceramic heating body, and an electronic vaporization device.

### BACKGROUND

A vaporizer is a device that vaporizes an aerosol-generation substrate into an aerosol, and is widely used in medical equipment and electronic vaporization devices. At present, the vaporizer generally adopts cotton core, fiber rope, or ceramic heating body to vaporize the aerosol-generation substrate, where a porous ceramic heating body is most widely used.

The operating principle of the porous ceramic heating body using e-liquid is mainly to use porous ceramic to absorb the e-liquid to a heating wire, and the heating wire heats to evaporate the e-liquid, to produce substances such as nicotine. However, in the existing ceramic heating body, one side of the ceramic substrate away from the heating wire is low in temperature, leading to a slow e-liquid guiding rate of the high-viscosity aerosol-generation substrate, resulting in insufficient e-liquid supply.

### SUMMARY

In an embodiment, the present invention provides a ceramic substrate. A thickness of the ceramic substrate ranges from 1 to 4 mm, and a thermal conductivity of the ceramic substrate ranges from 0.8 to 2.5 W/m·k.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a variation curve of the viscosity of different aerosol-generation substrates with the temperature.

FIG. 2 is a 2D variation diagram of the average temperature of one side of a ceramic substrate away from a heating element with the thermal conductivity when the vaporization temperature of the ceramic substrate is 350° C.

FIG. 3 is a 2D variation diagram of the smoke amount with the thermal conductivity of the ceramic substrate.

FIG. 4 is a time-variation curve of the temperature of one side of the ceramic substrate with the thermal conductivity of 1.3 W/m k away from the heating element under different powers.

### DETAILED DESCRIPTION

In an embodiment, the present invention overcomes the defect of the slow e-liquid guiding rate of the high-viscosity aerosol-generation substrate in the related art, and therefore, a ceramic substrate, ceramic heating body, and an electronic vaporization device are provided. In an embodiment, the present invention provides a ceramic substrate. The thickness of the ceramic substrate ranges from 1 to 4 mm, and the thermal conductivity ranges from 0.8 to 2.5 W/m·k.

Optionally, the thickness of the ceramic substrate ranges from 1.5 to 3 mm.

Optionally, the thermal conductivity of the ceramic substrate ranges from 1.0 to 2.0 W/m·k.

Optionally, the porosity of the ceramic substrate ranges from 40% to 70%, preferably 50% to 60%. Optionally, the ceramic substrate includes silicon carbide, aluminum oxide, and silicon dioxide, where
the weight percentage of the silicon carbide ranges from 10% to 70%; the weight percentage of the aluminum oxide ranges from 6% to 65%; and the weight percentage of the silicon dioxide ranges from 15% to 50%.

Optionally, the weight percentage of the silicon carbide ranges from 30% to 45%; the weight percentage of the aluminum oxide ranges from 40% to 55%; and the weight percentage of the silicon dioxide ranges from 15% to 50%.

Optionally, the pore size of the ceramic substrate ranges from 10 to 35 μm. Optionally, the ceramic substrate is a sheet structure.

A ceramic heating body is provided, including: the ceramic substrate described above, and
a heating element arranged on the ceramic substrate.

Optionally, the ceramic substrate includes a liquid absorbing surface, and the temperature of the liquid absorbing surface is greater than or equal to 80° C. during operation of the heating element.

An electronic vaporization device is provided, including the ceramic substrate and the ceramic heating body described above.

In this application, by selecting specific thickness and thermal conductivity, the heat generated by the heating element can be effectively conducted in the ceramic substrate, to raise the temperature (which may reach 80° C. or above) of the side of the ceramic substrate away from the heating element, so that the viscosity of the high-viscosity aerosol-generation substrate is reduced, and the high-viscosity aerosol-generation substrate has good fluidity. The cooperation between the thickness and the thermal conductivity resolves the problem of insufficient e-liquid supply easily caused by the slow e-liquid guiding rate of the high-viscosity aerosol-generation substrate.

During inhalation of an electronic vaporization device using a porous ceramic heating body, such as an electronic cigarette, a porous ceramic is mainly used to absorb an e-liquid to a heating element, and the heating element heats to evaporate the e-liquid, to produce substances such as nicotine. The porous ceramic heating body generally includes a ceramic substrate and a heating element arranged on a side surface of the ceramic substrate. The ceramic substrate has a vaporization surface and a liquid absorbing surface that are arranged opposite to each other. The liquid absorbing surface is used to absorb an aerosol-generation substrate, the vaporization surface is configured to vaporize the aerosol-generation substrate on the ceramic substrate, and the heating element is arranged on the vaporization surface side of the ceramic substrate. The ceramic substrate absorbs the e-liquid, and uses a capillary force to absorb the e-liquid to the heating element to be vaporized into smoke. However, two sides of the existing ceramic substrate are different in temperature. The temperature of one side in contact with the heating element is higher, and the temperature of the side away from the heating element is lower, leading to a slow e-liquid guiding rate of the high-viscosity aerosol-generation substrate, resulting in insufficient e-liquid supply.

The inventor found that one of the causes to the above phenomenon is the low thermal conductivity of the ceramic substrate, which makes heat generated by the heating element fail to be effectively conducted in the ceramic substrate, leading to low temperature of the ceramic substrate away from the heating element, which makes unsmooth e-liquid guiding of the high-viscosity aerosol-generation substrate, leading to a slow e-liquid guiding rate, resulting in insufficient e-liquid supply.

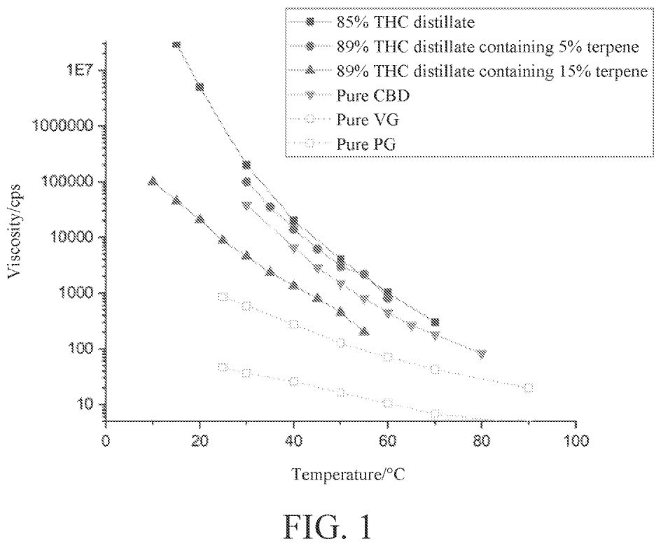

FIG. 1 is a variation curve of the viscosity of different aerosol-generation substrates with the temperature. Compared with a conventional aerosol-generation substrate such as an e-liquid, pure PG (Propylene Glycol), and pure VG (Vegetable Glycerin), the high-viscosity aerosol-generation substrate has high viscosity and poor fluidity at room temperature. Therefore, in a case that the ceramic substrate has low thermal conductivity, the temperature of the ceramic substrate away from the heating element is low. Due to the high viscosity of the aerosol-generation substrate, it easily leads to a slow e-liquid guiding rate of the aerosol-generation substrate in the ceramic heating body, resulting in insufficient e-liquid supply during inhalation. As shown in FIG. 1, the viscosity of the aerosol-generation substrate decreases rapidly as the temperature rises. Therefore, as long as the temperature of the ceramic substrate away from the heating element can be increased to maintain the high temperature on the two sides of the ceramic substrate, the viscosity of the aerosol-generation substrate can be reduced, the e-liquid guiding rate is ensured, and the insufficient e-liquid supply is avoided.

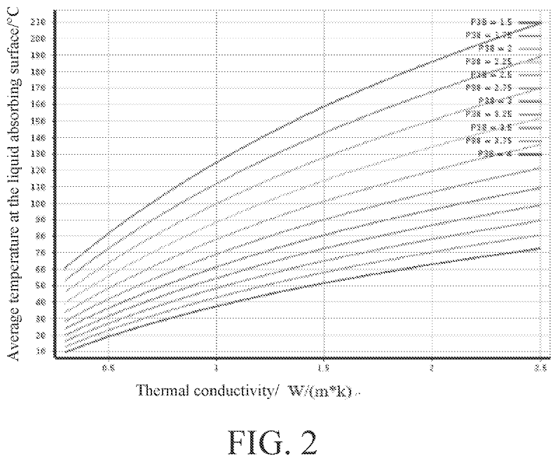

When the vaporization temperature of the ceramic substrate is 350° C., the variation of the average temperature of the side of the ceramic substrate away from the heating element with the thermal conductivity is shown in FIG. 2. In FIG. 2, the ceramic substrate includes silicon carbide, aluminum oxide, and silicon dioxide, where the weight percentage of the silicon carbide ranges from 10% to 70%; the weight percentage of the aluminum oxide ranges from 6% to 65%; the weight percentage of the silicon dioxide ranges from 15% to 50%; and the porosity of the ceramic substrate ranges from 50% to 60%. There are 11 curves in FIG. 2, which respectively represent the thermal conductivity-average temperature at the liquid absorbing surface curves of 11 ceramic substrates with different thicknesses, where P38 represents the thickness of each ceramic substrate. The thicknesses of the ceramic substrates respectively represented by the 11 forward curves from the origin of the coordinate axis to the y axis in sequence are: 4 mm, 3.75 mm, 3.5 mm, 3.25 mm, 3 mm, 2.75 mm, 2.5 mm, 2.25 mm, 2 mm, 1.75 mm, and 1.5 mm. It can be seen that when the thermal conductivity is 0.4 W/m·k, the average temperature of the side of the ceramic substrate away from the heating element (that is, the liquid absorbing surface) only ranges from 10 to 60° C. For example, when the thickness of the ceramic substrate is 2 mm, the average temperature of the side of the ceramic substrate away from the heating element is about 50° C. Therefore, only when the thermal conductivity of the ceramic substrate is controlled to a certain extent, the temperature of the ceramic substrate away from the heating element can reach the expected temperature.

In addition, as shown in FIG. 2, the temperatures on the side of the ceramic substrates with different thicknesses away from the heating element are also different under the same thermal conductivity. The thickness and the thermal conductivity of the ceramic substrate affect the temperature of the side of ceramic substrate away from the heating element.

Based on the above research, the inventor unexpectedly found that selecting an appropriate combination of the thickness and the thermal conductivity of the ceramic substrate can resolve the above technical problem, and therefore this application is completed.

According to an aspect of this application, a ceramic substrate is provided. The thickness of the ceramic substrate ranges from 1 to 4 mm, and the thermal conductivity ranges from 0.8 to 2.5 W/m·k.

In this application, by selecting specific thickness and thermal conductivity, the heat generated by the heating element can be effectively conducted in the ceramic substrate, to raise the temperature (which may reach 80° C. or above) of the side of the ceramic substrate away from the heating element, so that the viscosity of the high-viscosity aerosol-generation substrate is reduced, and the high-viscosity aerosol-generation substrate has good fluidity. The cooperation between the thickness and the thermal conductivity resolves the problem of insufficient e-liquid supply easily caused by the slow e-liquid guiding rate of the high-viscosity aerosol-generation substrate.

The thickness of the ceramic substrate refers to the vertical distance between the vaporization surface and the liquid absorbing surface of the ceramic substrate. The thickness of the ceramic substrate ranges from 1 to 4 mm, such as 1.2 mm, 1.5 mm, 1.8 mm, 2.1 mm, 2.4 mm, 2.7 mm, 3.0 mm, 3.3 mm, 3.6 mm, 3.9 mm, or 4.0 mm Viewing from ceramic strength and preparation technology, the thickness of the ceramic substrate is selected from 1.5 to 3 mm.

The thermal conductivity of the ceramic substrate ranges from 0.8 to 2.5 W/m·k, such as 0.8 W/m·k, 1.0 W/m·k, 1.2 W/m·k, 1.4 W/m·k, 1.6 W/m·k, 1.8 W/m·k, 2 W/m·k, or 2.5 W/m·k. If the thermal conductivity of the ceramic substrate is less than 0.8 W/m·k, the temperature of the side of the ceramic substrate away from the heating element cannot reach the expected temperature (80° C. or above), if the thermal conductivity of the ceramic substrate is greater than 2.5 W/m·k, the smoke amount does not meet the inhalation requirement. Considering from the perspective of maintaining a certain smoke amount, the thermal conductivity of the ceramic substrate ranges from 1.0 to 2.0 W/m·k.

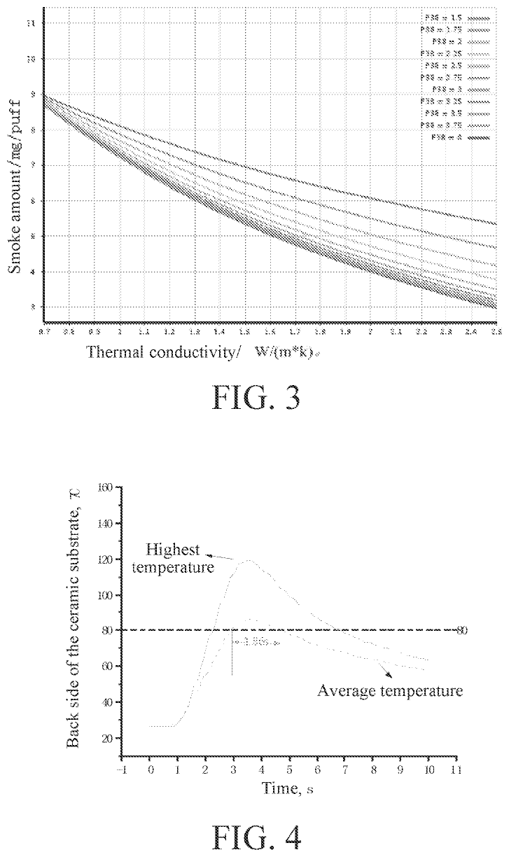

It should be noted that in this application, a test method for the thermal conductivity is ISO22007-2.2. The variation of the smoke amount with the thermal conductivity of the ceramic substrate is shown in FIG. 3. In FIG. 3, the ceramic substrate includes silicon carbide, aluminum oxide, and silicon dioxide, where the weight percentage of the silicon carbide ranges from 10% to 70%; the weight percentage of the aluminum oxide ranges from 6% to 65%; the weight percentage of the silicon dioxide ranges from 15% to 50%; and the porosity of the ceramic substrate ranges from 50% to 60%. P38 represents the thickness of each ceramic substrate. There are 11 curves in FIG. 3, which respectively represent the thermal conductivity and smoke amount curves of 11 ceramic substrates with different thicknesses. The thicknesses of the ceramic substrates respectively represented by the 11 forward curves from the origin of the coordinate axis to the y axis in sequence are: 4 mm, 3.75 mm, 3.5 mm, 3.25 mm, 3 mm, 2.75 mm, 2.5 mm, 2.25 mm, 2 mm, 1.75 mm, and 1.5 mm. It can be seen that when the thermal conductivity is 2.2 W/m·k, the average smoke amount is
3.7 to 5.8 mg/puff. For example, when the thickness of the ceramic substrate is 2 mm, the average smoke amount is 4.7 mg/puff. Therefore, in this application, by selecting specific thermal conductivity, a high average smoke amount that can reach ≥4.5 mg/puff is also achieved, achieving the expected inhalation experience.

It should be noted that, in this application, a test method for the smoke amount is:
to use a smoke inhalation machine and set the inhalation capacity to 60 ml. Each puff takes 3 s and stops for 30 s. Before the experiment, a balance is used to weight a cartridge. After every 10 puffs, the cartridge is re-weighted. The difference between the two is divided by 10 to obtain the average smoke amount of each puff.

In an optional embodiment, the porosity of the ceramic substrate ranges from 40% to 70%, such as 40%, 45%, 50%, 55%, 60%, 65%, or 70%. If the porosity is less than 40%, the liquid amount of the e-liquid delivered to the heating element is affected, and problems such as dry heating or smell of scorching may occur. If the porosity is greater than 70%, the strength of the ceramic substrate is affected, which is not conducive to improving the service life of a vaporization core. Considering from the perspective of the e-liquid delivery and the strength of the ceramic substrate, the porosity of the ceramic substrate ranges from 50% to 60%.

It should be noted that, in this application, a test method for the porosity is: “Part 3 of ceramic tile test method GB/T3810.3-2016: Water Absorption, Apparent Relative Density of Apparent Porosity and Part: Determination of Water Absorption, Apparent Relative Density of Apparent Porosity and Bulk Density”.

In this application, the high-viscosity aerosol-generation substrate refers to an aerosol-generation substrate with the viscosity greater than 10000 cps at room temperature (25° C.).

It should be noted that, in this application, a determination method for the viscosity is: GBT17473.5-1998 test method for precious metal paste for thick film microelectronics.

FIG. 4 is a time-variation curve of the temperature of the back side of the ceramic substrate in a cuboid sheet structure with the thermal conductivity of 1.3 W/m·k, the thickness of 2 mm, and the porosity of 57% (that is, the temperature of one side of the ceramic substrate away from the heating element) under different powers. The ceramic substrate includes silicon carbide 18 wt %, aluminum oxide 43.2 wt %, and silicon dioxide 34.9 wt %. In the high-viscosity aerosol-generation substrate, the backside temperature of the ceramic substrate under different powers is shown in FIG. 4 during inhalation of a user, wherein the solid line is the highest temperature of the back side of the ceramic substrate at different time, and the dashed line is the average temperature of the backside of the ceramic substrate at different time. It can be seen from FIG. 4 that, the average temperature on the back side of the ceramic substrate can reach 80° C. or above during inhalation. 80° C. can provide a good e-liquid guiding environment for the high-viscosity aerosol-generation substrate, and the e-liquid guiding rate is better.

In an optional embodiment, the ceramic substrate includes silicon carbide, aluminum oxide, and silicon dioxide, where the weight percentage of the silicon carbide ranges from 10% to 70%; the weight percentage of the aluminum oxide ranges from 6% to 65%; and the weight percentage of the silicon dioxide ranges from 15% to 50%. In an optional embodiment, the weight percentage of the silicon carbide ranges from 30% to 45%, the weight percentage of the aluminum oxide ranges from 40% to 55%; and the weight percentage of the silicon dioxide ranges from 15% to 20%. In an optional embodiment, the ceramic substrate further includes additives. The weight percentage of the additives ranges from 0% to 10%, and the additives are, for example, reinforcers and adhesives.

In an optional embodiment, a preparation method for the ceramic substrate includes:
obtaining silicon carbide powder with the weight percentage from 10% to 70% of, aluminum oxide powder with the weight percentage from 6% to 65%, and silicon dioxide powder with the weight percentage from 15% to 50% and mixing all. Specifically, silicon carbide powder with the weight percentage from 10% to 70% of, aluminum oxide powder with the weight percentage from 6% to 65%, and silicon dioxide powder with the weight percentage from 15% to 50% are respectively weighted in the same container. Then water is added into the container and stirred to mix the water with the silicon carbide, aluminum oxide and silicon dioxide powder. The mixing and stifling time may range from 15 to 30 minutes, and optionally, 20 to 25 minutes. Optionally, the weight percentage of the silicon carbide powder may range from 30% to 45%; the weight percentage of the aluminum oxide powder may range from 40% to 55%; and the weight percentage of the silicon dioxide powder may range from 15% to 20%.

The mixed powder is pressed and formed to obtain a ceramic green body. In a specific embodiment, the mixed powder may be first put into equipment such as a drying oven for drying. Then the dried powder is granulated in a manner such as spraying and stirring. Next, the granulated particles are put into a mold, and the granulated particles are hot pressed and dry pressed by a dry pressing machine under a preset pressure, to obtain the ceramic green body. The preset pressure specifically ranges from 10 to 40 MPa. The mold is specifically used to prepare a ceramic heating substrate for the vaporization core.

The raw ceramic green body is sintered and cooled at a preset temperature, to obtain the ceramic substrate. Specifically, the preset temperature may range from 1100 to 1700° C., and the temperature holding time ranges from 2 to 8 hours. Optionally, the preset temperature may range from 1200 to 1500° C., and the temperature holding time ranges from 2 to 4 hours.

In an optional embodiment of this application, the ceramic substrate is a sheet structure, and the sheet structure may be a rectangular, circular or oval sheet structure, or the sheet structure may be a flat or curved structure.

In an optional embodiment of this application, the pore size of the ceramic substrate ranges from 10 to 35 μm. The pore size in this range can ensure the e-liquid supply amount and the e-liquid supply speed of the ceramic substrate.

According to another aspect of this application, a ceramic heating body is provided, including the ceramic substrate described above, and a heating element arranged on the ceramic substrate.

The ceramic heating body is configured to heat and vaporize a high-viscosity aerosol-generation substrate when powered on, the heating element is configured to generate heat when powered on, and the ceramic substrate conducts heat for the heat generated by the heating element.

Optionally, the ceramic substrate includes a liquid absorbing surface, and the temperature of the liquid absorbing surface is greater than or equal to 80° C. during operation of the heating element. The liquid absorbing surface is one side of the ceramic substrate away from the heating element.

Specifically, the ceramic substrate has a vaporization surface and a liquid absorbing surface that are arranged opposite to each other. The liquid absorbing surface is used to absorb an aerosol-generation substrate, the vaporization surface is configured to vaporize the aerosol-generation substrate on the ceramic substrate, and the heating element is arranged on the vaporization surface side of the ceramic substrate. A typical but non-restrictive heating element is, for example, a metal heating wire. The ceramic heating body includes the ceramic substrate described above, which can achieve the same or similar technical effects, which are not repeated herein.

According to another aspect of this application, an electronic vaporization device is provided, including the ceramic substrate or the ceramic heating body described above. The electronic vaporization device includes the ceramic substrate described above, which can achieve the same or similar technical effects, which are not repeated herein. The electronic vaporization device is, for example, an electronic cigarette.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A ceramic substrate, wherein a thickness of the ceramic substrate ranges from 1 to 4 mm, and a thermal conductivity of the ceramic substrate ranges from 0.8 to 2.5 W/m·k.

2. The ceramic substrate of claim 1, wherein the thickness of the ceramic substrate ranges from 1.5 to 3 mm.

3. The ceramic substrate of claim 1, wherein the thermal conductivity of the ceramic substrate ranges from 1.0 to 2.0 W/m·k.

4. The ceramic substrate of claim 1, wherein a porosity of the ceramic substrate ranges from 40% to 70%.

5. The ceramic substrate of claim 1, further comprising:
silicon carbide, aluminum oxide, and silicon dioxide,
wherein a weight percentage of the silicon carbide ranges from 10% to 70%,
wherein a weight percentage of the aluminum oxide ranges from 6% to 65%, and
wherein a weight percentage of the silicon dioxide ranges from 15% to 50%.

6. The ceramic substrate of claim 5, wherein the weight percentage of the silicon carbide ranges from 30% to 45%,
wherein the weight percentage of the aluminum oxide ranges from 40% to 55%, and
wherein the weight percentage of the silicon dioxide ranges from 15% to 50%.

7. The ceramic substrate of claim 1, wherein a pore size of the ceramic substrate ranges from 10 to 35 μm.

8. The ceramic substrate of claim 1, wherein the ceramic substrate comprises a sheet structure.

9. A ceramic heating body, comprising:
the ceramic substrate of claim 1; and
a heating element arranged on the ceramic substrate.

10. The ceramic heating body of claim 9, wherein the ceramic substrate comprises a liquid absorbing surface, and
wherein, during operation of the heating element, a temperature of the liquid absorbing surface is greater than or equal to 80° C.

11. An electronic vaporization device, comprising:
the ceramic heating body of claim 9.

12. The ceramic substrate of claim 4, wherein the porosity of the ceramic substrate ranges from 50% to 60%.
